(19) United States
(12) Patent Application Publication
(10) Pub. No.: US 2018/0098924 A1
(43) Pub. Date: Apr. 12, 2018

(54) COSMETIC PRODUCT INCLUDING ETHANOL AND AN AMYLATE IN A DEVICE FOR FLASH EVAPORATION

(71) Applicant: Henkel AG & Co. KGaA, Duesseldorf (DE)

(72) Inventors: Thorsten Knappe, Schenefeld (DE); Rolf Bayersdoerfer, Humburg (DE); Thomas Foerster, Duesseldorf (DE)

(21) Appl. No.: 15/615,034

(22) Filed: Jun. 6, 2017

Related U.S. Application Data
(63) Continuation of application No. PCT/EP2015/075374, filed on Nov. 2, 2015.

(30) Foreign Application Priority Data
Dec. 10, 2014 (DE) 10 2014 225 421.3

(51) Int. Cl. A61K 8/04; A61K 8/34; A61K 8/73; A61Q 5/02; A61Q 5/06

(52) U.S. Cl. CPC A61K 8/046; A61K 8/34; A61K 8/732; A61K 2800/81; A61Q 5/06; A61K 2800/87; A61Q 5/02

(57) ABSTRACT

The invention relates to a cosmetic product comprising a) a cosmetic preparation including, in relation to the total weight a1) 20-98% by weight ethanol, a2) 5-80% by weight of at least one amylate, b) a device for flash evaporation of the cosmetic preparation a), and to a method using corresponding products and the use of the cosmetic preparation a) as process material in a device for flash evaporation.

### FIELD OF THE INVENTION

The present invention generally relates to the technical field of the treatment of keratin-containing fibers, in particular human hair. The subject of the invention concerns specific hair cosmetic formulations, which are suitable for application to keratin-containing fibers by means of a flash evaporation method. Moreover, the use of said hair cosmetic formulations in devices for flash evaporation and methods for hair treatment are the subject of the present invention.

### BACKGROUND OF THE INVENTION

Today, an attractive hairstyle is generally regarded as an essential part of a well-groomed appearance. An essential aspect of hair care is the cleaning of hair.

In the field of the cleaning and care of keratinic fibers, the spray application of suitable cosmetic preparations is of certain importance, wherein the preparations are usually applied as pump sprays or aerosol sprays. For this purpose, the cosmetic preparations are packaged in a dispensing device, from which they are sprayed either by mechanical force or with the aid of a propellant via a valve. Both methods have obvious disadvantages. Whereas pump sprays are generally not suitable for a long-lasting uniform spray application of hair cosmetic preparations, aerosol sprays are based on the use of propellants or propellant gases, which, on the one hand, exert no cosmetic effect and which, on the other, can pose a danger to the user when handled improperly.

Against this background, there is a need for alternative approaches to the spraying of hair cosmetic preparations. Flash evaporation has proven to be an alternative spray method of this kind. In this method, which is described, for example, in the international patent application WO 200183071 A1 (Henkel), a liquid or paste-form, solvent-containing composition is heated in an enclosed space to a temperature above the boiling point of the solvent, as a result of which excess pressure is created in the composition. Upon release (throttling) of the pressure, the liquid evaporates and can then be atomized, for example, by a suitable nozzle.

Even if flash evaporation is, therefore, basically suitable for the spray application of hair cosmetic preparations, thus at the same time not every hair cosmetic preparation agent can be atomized by a flash evaporation method. This is due, on the one hand, to the heating of the cosmetic preparation necessary for flash evaporation and, on the other, to the specifics of the spray mist produced by flash evaporation, for example, the produced droplet size and droplet density in the spray mist.

### BRIEF SUMMARY OF THE INVENTION

An object of the present invention, therefore, was to provide specific hair cosmetic preparations for the cleaning and care of keratin-containing fibers, which based on their chemical and physical properties are suitable for the targeted spray application using a device for flash evaporation. Furthermore, the preparations are to be suitable for realizing a good cleaning and care effect after application using a flash method. It emerged that of the many known preparations effective as hair cosmetics, alcoholic starch preparations in particular are suitable for achieving said object.

A first subject of the present invention, therefore, is a cosmetic product comprising
a) a cosmetic preparation including, based on its total weight,
   a1) 20 to 95% by weight of ethanol,
   a2) 5 to 80% by weight of at least one starch compound;
b) a device for the flash evaporation of cosmetic preparation a).

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

Cosmetic preparation a) is preferably liquid. Cosmetic preparation a) can be present as a solution or dispersion, for example, as an emulsion or suspension. Preferred cosmetic preparations a) are present in the form of a solution or suspension.

The cosmetic preparation of the invention includes 20 to 98% by weight of ethanol as the first essential component. Preferred cosmetic products are characterized in that the weight proportion of ethanol a1) in terms of the total weight of cosmetic preparation a) is 30 to 90% by weight, preferably 40 to 80% by weight. Corresponding agents are characterized by a good cosmetic effect with simultaneously good appliability.

A second essential component of the cosmetic compositions of the invention is starch compound a2). It has proven advantageous in regard to the producibility, appliability, and cosmetic effect of cosmetic preparations of the invention, if the weight proportion of the starch compound in terms of the total weight of cosmetic preparation a) is 10 to 70% by weight, preferably 20 to 60% by weight. Surprisingly, the high weight proportion of the starch compound according to the invention can also be easily sprayed by the device for flash evaporation. This succeeds in particular also without a possibly employed nozzle becoming clogged.

The second essential component of cosmetic preparations a) of the invention is starch compound a1). The term “starch” in this case is understood to mean a storage carbohydrate, which is stored by many plants in the form of starch grains (granules) usually 1 to 200 μm in size in different plant parts, e.g., in tubers or roots, grain seeds, fruit, and in marrow.

Preferably, starch compound a2) is present as solid particles dispersed in ethanol a1). “Solid particles” are understood to mean solids that are particulate at 20° C. and 1013.25 mbar.

A starch compound preferably usable according to the invention is selected from at least one, optionally modified, polycondensation product of D-glucose obtained from the starch of potatoes, corn, rice, peas, acorns, chestnuts, barley, wheat, bananas, sago, millet, sorghum, oats, barley, rye, beans, sweet potato, arrowroot, or cassava. Particularly preferably, the agent of the invention includes at least one starch compound, which is tapioca starch, potato starch, corn starch, or rice starch or is derived therefrom. Mixtures of the aforementioned starch compounds are also included according to the invention.

The starch compound is very particularly preferably rice starch. Starch compounds based on rice starch are obtainable, for example, under the name Remy DR KA (INCI name: Oryza Sativa (Rice) Starch, CAS number 9005-25-8) from the company Bene O Remy Industries or under the name Rice Starch D.S.A. 7 (INCI name: Oryza Sativa (Rice) Starch, Cetrimonium Chloride; CAS number 9005-25-8) from the company Agrana.

The cosmetic products of the invention, furthermore, comprise, apart from cosmetic preparation a), a device for flash evaporation. The term “flash evaporation” in the context of the present invention describes the creation of vapor when pressure is reduced in a closed space filled with a liquid and having an excess pressure (relative to the environment). A suitable excess pressure can be produced, for example, by heating an amount of cosmetic preparation a) to a temperature T₁ in an enclosed space. In the closed space, the liquid at the particular temperature T₁ has a pressure, the saturation pressure p₁. If the closed space is opened, for example, by a valve to a relaxation space that is not at excess pressure but has the pressure p₀<p₁, then the pressure in the previously closed space is reduced and cosmetic preparation a) or the solvent included in the cosmetic preparation or parts of said solvent evaporate within the scope of the spread of the new pressure level. The forming vapor or spray mist can be used for applying specific cosmetic preparations.

If cosmetic preparation a) is therefore heated in a closed space proceeding from standard conditions (T₀=25° C., p₀=1.000 bar), thus an increased pressure of cosmetic preparation a) also results, in addition to an increased temperature. This increased pressure can be relieved in a relaxation space to a pressure p₀, for example, the ambient air pressure (p₀=1.000 bar), as a result of which evaporation of cosmetic preparation a) is achieved at least partially.

The pressure of cosmetic preparation a) can be relieved directly in the space in which it was previously heated. However, the heated cosmetic preparation a) at excess pressure can be transported alternatively after heating as well to a second chamber in which the pressure release then occurs.

Flash evaporation, in other words, is a method in which cosmetic preparation a) is heated by a heating device in a closed container to temperatures above the ambient temperature, wherein a pressure above the ambient pressure forms in the container and the heated cosmetic preparation a) under increased pressure is then released from the container into the environment.

A device for flash evaporation accordingly is a device that comprises a container and a heating device and is designed such that a cosmetic preparation a) can be heated in the closed container by the heating device to temperatures above the ambient temperature in a way that a pressure above the ambient pressure arises in the container and the heated cosmetic preparation a) under increased pressure can be released from the container into the environment.

Concurrently with or after the pressure release, cosmetic preparation a) can be supplied to a nozzle which can influence, for example, the properties of the vapor or spray mist produced by the flash evaporation, in particular the droplet size or the droplet density but also the spray width and the shape of the spray cone. The use of nozzles, preferably atomizing nozzles, is therefore preferable. The specific nozzle type or the specific nozzle design is selectively established depending on the particular spray mist properties.

In summary, a preferred device for flash evaporation has
b1) a container b1) which is to be opened and closed by means of a valve and defines the closed interior space in which the cosmetic preparation can be taken up,
b2) a heating device b2) which makes it possible to heat the cosmetic preparation located in container b1).

Particularly preferred is the use of an additional nozzle b3), which enables an atomization of cosmetic preparation a) escaping from the container. As an alternative to a valve, a similarly acting closing element, which can close or release an associated opening in the container by an appropriate change in position, can also be used.

A preferred subject of the present invention is a cosmetic product comprising
a) a cosmetic preparation including, based on its total weight,
   a1) 20 to 95% by weight of ethanol,
   a2) 5 to 80% by weight of at least one starch compound;
b) a device for the flash evaporation of cosmetic preparation a), wherein the device for flash evaporation comprises a container b1) and a heating device b2 and is designed such that
   cosmetic preparation a) can be taken up in the interior space of container b1),
   the interior space of container b1), said space filled at least partially with cosmetic preparation a), can be closed,
   cosmetic preparation a) can be heated in the closed interior space of container b1) by means of heating device b2) with an increase in pressure.

A particularly preferred subject of the present invention, therefore, is a cosmetic product comprising
a) a cosmetic preparation including, based on its total weight,
   a1) 20 to 95% by weight of ethanol,
   a2) 5 to 80% by weight of at least one starch compound;
b) a device for the flash evaporation of cosmetic preparation a), comprising
   b1) a container b1) to be closed and opened by means of a valve,
   b2) a heating device which makes it possible to heat the cosmetic preparation located in container b1),
   b3) a nozzle b3) which enables an atomization of cosmetic preparation a).

A particularly preferred subject of the present invention, in other words, is a cosmetic product comprising
a) a cosmetic preparation including, based on its total weight,
   a1) 20 to 95% by weight of ethanol,
   a2) 5 to 80% by weight of at least one starch compound;
b) a device for the flash evaporation of cosmetic preparation a), wherein the device for flash evaporation comprises a container b1) and a heating device b2 and is designed such that
   cosmetic preparation a) can be taken up in the interior space of container b1),
   the interior space of container b1), said space filled at least partially with cosmetic preparation a), can be closed,
   cosmetic preparation a) can be heated in the closed interior space of container b1) by means of heating device b2) with an increase in pressure,
   the heated cosmetic preparation a) can be released from the interior space of container b1) into the environment with a reduction in pressure.

Container b1), in which the cosmetic preparation is heated, is designed in a way that makes it possible to close off completely said container from the environment during the heating of cosmetic preparation a) and to open it after the heating, so as to enable flash evaporation of cosmetic preparation a). This can be assured, for example, by a structural element for flow control, in particular a valve.

Container b1), in which cosmetic preparation is heated, is preferably in contact with another container from which the cosmetic preparation amount provided for flash evaporation is transferred before heating into container b1). The access between this reservoir and container b1) in this case is to be opened and closed by a suitable device, for example, a valve. This additional container is preferably made in the form of a reservoir; i.e., it comprises preferably a multiple, for example, more than 10 times, preferably more than 50 times, of the amount of the cosmetic preparation necessary for an evaporation process. In other words, the additional container/reservoir preferably has a multiple, for example, more than 10 times the volume, preferably more than 20 times, and in particular more than 50 times the volume of container b1).

A further particularly preferred subject of the present invention, therefore, is a cosmetic product comprising
a) a cosmetic preparation including, based on its total weight,
   a1) 20 to 95% by weight of ethanol,
   a2) 5 to 80% by weight of at least one starch compound;
b) a device for the flash evaporation of cosmetic preparation a), comprising
   b1) a container b1) to be closed and opened by means of a valve,
   b2) a heating device which makes it possible to heat the cosmetic preparation located in closed container b1),
   b3) a nozzle b3) which enables an atomization of cosmetic preparation a);
c) a reservoir for cosmetic preparation a), from which cosmetic preparation a) can enter into container b1), wherein
   the access between the reservoir and container b1) has a structural element for flow regulation by which the flow of cosmetic preparation a) from the reservoir into container b1) can be interrupted;
   the reservoir has at least 10 times the volume, preferably at least 20 times, and in particular at least 50 times the volume of container 1).

The reservoir is not a pressurized container and the cosmetic composition in the reservoir is not under pressure; in other words, the pressure in the interior of the reservoir corresponds to the ambient pressure (also air pressure or atmospheric pressure). Thus, corresponding cosmetic products do not comprise, for example, any propellant. The cosmetic product also does not have a pump device, which is suitable for releasing or spraying the cosmetic preparation into the environment without the action of the device for flash evaporation.

A very particularly preferred subject of the present invention, therefore, is a cosmetic product comprising
a) a cosmetic preparation including, based on its total weight,
   a1) 20 to 95% by weight of ethanol,
   a2) 5 to 80% by weight of at least one starch compound;
b) a device for the flash evaporation of cosmetic preparation a), comprising
   b1) a container b1) to be closed and opened by means of a valve,
   b2) a heating device which makes it possible to heat the cosmetic preparation located in closed container b1),
   b3) a nozzle b3) which enables an atomization of cosmetic preparation a);
c) a reservoir for cosmetic preparation a), from which cosmetic preparation a) can enter into container b1), wherein
   the access between the reservoir and container b1) has a structural element for flow regulation by which the flow of cosmetic preparation a) from the reservoir into container b1) can be interrupted;
   the reservoir has at least 10 times the volume, preferably at least 50 times the volume of container b1;
   the pressure in the interior of the reservoir corresponds to the ambient pressure.

A very particularly preferred subject of the present invention, therefore, is a cosmetic product comprising
a) a cosmetic preparation including, based on its total weight,
   a1) 20 to 95% by weight of ethanol,
   a2) 5 to 80% by weight of at least one starch compound;
b) a device for the flash evaporation of cosmetic preparation a), comprising
   b1) a container b1) to be closed and opened by means of a valve,
   b2) a heating device which makes it possible to heat the cosmetic preparation located in closed container b1),
   b3) a nozzle b3) which enables an atomization of cosmetic preparation a);
c) a reservoir for cosmetic preparation a), from which cosmetic preparation a) can enter into container b1), wherein
   the access between the reservoir and container b1) has a structural element for flow regulation by which the flow of cosmetic preparation a) from the reservoir into container b1) can be interrupted;
   the reservoir has at least 10 times the volume, preferably at least 50 times the volume of container b1;
   the pressure in the interior of the reservoir corresponds to the ambient pressure and the cosmetic product comprises no propellant.

Preferred furthermore are cosmetic products, comprising
a) a cosmetic preparation including, based on its total weight,
   a1) 20 to 95% by weight of ethanol,
   a2) a2) 5 to 80% by weight of at least one starch compound;
b) a device for the flash evaporation of cosmetic preparation a), comprising
   b1) a container b1) to be closed and opened by means of a valve,
   b2) a heating device which makes it possible to heat the cosmetic preparation located in closed container b1),
   b3) a nozzle b3) which enables an atomization of cosmetic preparation a);
c) a reservoir for cosmetic preparation a), from which cosmetic preparation a) can enter into container b1), wherein
   the access between the reservoir and container b1) has a structural element for flow regulation by which the flow of cosmetic preparation a) from the reservoir into container b1) can be interrupted;
   the reservoir has at least 10 times the volume, preferably at least 50 times the volume of container b1;
   the pressure in the interior of the reservoir corresponds to the ambient pressure,
   wherein the cosmetic product does not have a pump device that is suitable for releasing or spraying cosmetic preparation a) without the action of the device for flash evaporation.

In summary, a particularly preferred subject of the present invention, therefore, is a cosmetic product comprising
a) a cosmetic preparation including, based on its total weight,
   a1) a1) 20 to 95% by weight of ethanol,
   a2) a2) 5 to 80% by weight of at least one starch compound;
b) a device for the flash evaporation of cosmetic preparation a), comprising
   b1) a container b1) to be closed and opened by means of a valve,
   b2) a heating device which makes it possible to heat the cosmetic preparation located in closed container b1),
   b3) a nozzle b3) which enables an atomization of cosmetic preparation a);
c) a reservoir for cosmetic preparation a), from which cosmetic preparation a) can enter into container b1), wherein
   the access between the reservoir and container b1) has a structural element for flow regulation by which the flow of cosmetic preparation a) from the reservoir into container b1) can be interrupted;
   the reservoir has at least 10 times the volume, preferably at least 50 times the volume of container b1;
   the pressure in the interior of the reservoir corresponds to the ambient pressure and the cosmetic product comprises no propellant,
      wherein the cosmetic product does not have a pump device that is suitable for releasing or spraying cosmetic preparation a) without the action of the device for flash evaporation.

Apart from the previously described components a1) and a2), cosmetic preparation a) can include further active or auxiliary substances, wherein active or auxiliary substances of this kind that improve the producibility, appliability, and/or the cosmetic action of the cosmetic preparations of the invention are preferred in particular.

A first example of a preferred active and auxiliary substance is cationic surfactants a3). Preferred cationic surfactants a3) are selected from quaternary ammonium compounds, esterquats, and amidoamines. The cationic surfactants are included in cosmetic preparation a), based on their total weight, in amounts of 0.01 to 4.0% by weight, preferably 0.05 to 2.0% by weight, and in particular 0.1 to 1.0% by weight. Cationic surfactants a3) from the group of quaternary ammonium compounds are particularly preferred.

Preferred quaternary ammonium compounds are ammonium halides, in particular chlorides and bromides, such as alkyltrimethylammonium chlorides, dialkyldimethylammonium chlorides, and trialkylmethylammonium chlorides, e.g., cetyltrimethylammonium chloride, stearyltrimethylammonium chloride, distearyldimethylammonium chloride, lauryldimethylammonium chloride, lauryldimethylbenzylammonium chloride, and tricetylmethylammonium chloride, and the imidazolium compounds known under the INCI names Quaternium-27 and Quaternium-83. The long alkyl chains of the aforesaid surfactants preferably have 10 to 18 carbon atoms. Very particularly preferred cosmetic preparations a) of the invention are characterized in that, based on their total weight, they include 0.01 to 4.0% by weight, preferably 0.05 to 2.0% by weight, and in particular 0.1 to 1.0% by weight (C₁₂ to C₁₈) alkyltrimethylammonium salt(s).

The hydrophobically modified metal oxide powder a4) form a second group of optional active and auxiliary substances. It has proven advantageous in this case in regard to the cosmetic action, if the weight proportion of the hydrophobically modified metal oxide powder in terms of the total weight of cosmetic preparation a) is 0.01 to 3.0% by weight, preferably 0.05 to 2.5% by weight, and in particular 0.1 to 2.0% by weight.

Metal oxides that have been modified at least on the surface of the particles such that the modified particle is wetted less by water than the unmodified particle are to be understood as “hydrophobically modified” or “hydrophobized” in the context of the invention. Silanized, hydrophobized metal oxides are particularly preferred. At least one representative of the group formed by silanes, halosilanes, alkoxysilanes, and silazanes is preferably suitable according to the invention as a reagent for silanizing the metal oxide. Preferably suitable hydrophobized metal oxides of the hydrophobized metal oxide powder are selected according to the invention from at least one representative of the group formed by hydrophobized silicates, hydrophobized aluminosilicates, hydrophobized titanium dioxide, and hydrophobized silicon dioxide. Hydrophobized silicates have proven to be particularly suitable for producing the cosmetic agents of the invention, whereby pyrogenic silicic acid aftertreated by silanization or by reaction with polydimethylsiloxane has particular advantages.

Cosmetic preparations a) of the invention preferably include hydrophobized silicon dioxide. The type of hydrophobically modified silicon dioxide a4) is not limited in principle, but because of the cosmetic properties of suitable agents preferably at least one silanized, hydrophobized silicon dioxide is used as the hydrophobized metal oxide powder.

Preferably, at least one representative of the group formed by silanes, halosilanes, alkoxysilanes, and silazanes is suitable according to the invention as a reagent for silanizing the silicon dioxide.

Preferred representatives of the group of silanes are hexa(C₁-C₂₀) alkyl disilanes, particularly hexamethyldisilane.

If a halosilane is used as the silylating agent, selected as the preferred halosilane is at least one compound from the group formed by the compounds
[(C₁-C₂₀)alkyl]_{z′}SiX_{(4-z′)}
X₃Si[(CH₂)ₙ—R]
X₂[(C₁-C₂₀)alkyl]Si(CH₂)ₙ—R
[(C₁-C₂₀)alkyl]_{(y′+1)}[R—(CH₂)ₙ]_{(2-y′)}SiX
where
X denotes a chlorine, bromine, or iodine atom,
z′ is a number 1, 2, or 3,
y′ is a number 0, 1, or 2,
n is an integer from 1 to 20, and
R stands for a group from
(C₁-C₁₀) alkyl-, aryl-, (C₁-C₆) perfluoroalkyl-, —NH₂, —N₃, —SCN, —CH=CH₂,

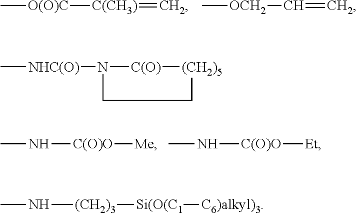

If an alkoxysilane is used as the silylating agent, selected as the preferred alkoxysilane is at least one compound from the group formed by the compounds
[(C1-C₂₀)alkylO]_{z}Si(C₁-C₂₀)alkyl_{(4-z)}
[(C₁-C₂₀)alkylO]_{z}Si[(CH₂)ₙ—-R]_{(4-z)}
[(C₁-C₂₀)alkylO]₂[(C₁-C₂₀)alkyl]Si(CH₂)ₙ—R
[(C₁-C₂₀)alkylO][(C₁-C₂₀)alkyl]₂Si(CH₂)n—R
[(C₁-C₂₀)alkylO][(C₁-C₂₀)alkyl]Si[(CH₂)ₙ—R]₂
(C₁-C₂₀alkyl)₃SiO—C(CH₃)═N—Si(C₁-C₂₀)alkyl₃,
where
n is an integer from 1 to 20, and
z denotes a number 1, 2, or 3,
R stands for a group from

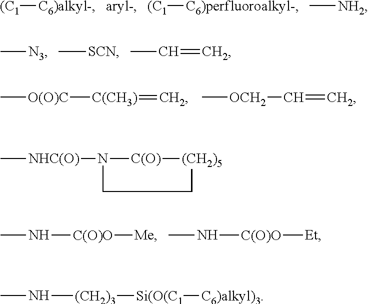

Selected as the preferred silazane is at least one compound from the class of disilazanes, in particular at least one compound from disilazanes of the formula

R′₂R″Si—NH—SiR′₂R″,
where
R′ denotes a (C₁-C₂₀) alkyl group and
R″ denotes a (C₁-C₂₀) alkyl group or a vinyl group. A particularly preferred silazane is hexamethyldisilazane.

All of the aforesaid alkyl groups, whether (C₁-C₆) alkyl, (C₁-C₁₀) alkyl, or (C₁-C₂₀) alkyl, can be both cyclic and linear or branched. Examples of alkyl groups usable according to the invention are methyl, ethyl, n-propyl, isopropyl, n-butyl, cyclopentyl, cyclohexyl, n-decyl, lauryl, myristyl, cetyl, stearyl, isostearyl, and behenyl.

An example of an aryl group of the invention is the phenyl group.

Examples of a (C₁-C₆) perfluoroalkyl group of the invention are trifluoromethyl, perfluoroethyl, perfluoropropyl, and perfluorohexyl.

Used preferably are hydrophobized silicon dioxides obtained by silanization of pyrogenic silicon dioxide.

Silanized, hydrophobized silicon dioxides are selected particularly preferably from at least one compound of the group formed by trimethyl silylate-coated silicon dioxide, dimethyl silylate-coated silicon dioxide, and octyl silylate-coated silicon dioxide.

Preferred cosmetic preparations are characterized in that the hydrophobically modified metal oxide powder a4) is selected from the group of hydrophobized silicates obtained by silanization of pyrogenic silicon dioxide.

A variety of suitable hydrophobically modified silicon dioxides are commercially available. Recited as examples are Aerosil® R104 V, Aerosil® R106, Aerosil® R202, Aerosil® R805, Aerosil® R812, Aerosil® R812S, Aerosil® R972, and Aerosil® R8200, all from Degussa, and HDK® H2000, HDK° H2050, and HDK® H3004, all from Wacker. It is very particularly preferred to use the silicon dioxide with the INCI name Silica Dimethyl Silylate, which is marketed by the company Degussa under the name Aerosil® R972. In summary, cosmetic preparations a) are preferred in which the hydrophobically modified metal oxide powder a1) is selected from the compounds with the INCI names: Silica Dimethyl Silylate (e.g., Aerosil R792, Aerosil R794), Silica Dimethicone Silylate (e.g., Aerosil R202), and Silica Silylate (e.g., Aerosil R805, Aerosil R812, Aerosil R816). Compounds with the INCI name Silica Dimethyl Silylate are particularly preferred.

The hydrophobically modified silicon dioxides with the INCI name Silica Dimethyl Silylate enable, in comparison with the customary previously described hydrophobically modified silicon dioxides, a simplified production of cosmetic agents of the invention, which are notable, moreover, for an improved appliability and cosmetic effect. The particularly preferred hydrophobized silicon dioxides with the INCI name Silica Dimethyl Silylate can be produced, for example, by reacting pyrogenic silicic acid with dimethyldichlorosilane. A particularly preferred subject of the present invention, therefore, is a cosmetic agent that includes a hydrophobically modified metal oxide powder, obtained by reacting pyrogenic silicic acid with dimethyldichlorosilane.

The particle diameter of the primary particles of preferred hydrophobically modified metal oxides a4), in particular the hydrophobically modified metal oxides with the INCI name Silica Dimethyl Silylate, is preferably less than 5 μm, particularly preferably less than 1 82 m, and in particular between 1 and 50 nm.

Preferred, furthermore, are hydrophobically modified metal oxides a4) of this kind, in particular hydrophobically modified metal oxides with the INCI name Silica Dimethyl Silylate, which have a specific BET surface between 10 and 400 m²/g, preferably between 40 to 300 m²/g, and in particular 80 to 150 m²/g.

In particular, additional care substances can be named as further suitable active or auxiliary substances.

The agent can include as a care substance, for example, at least one protein hydrolysate and/or a derivative thereof. Protein hydrolysates are product mixtures obtained by acid-, base-, or enzyme-catalyzed degradation of proteins. The term ‘protein hydrolysates’ according to the invention is also understood to mean total hydrolysates, as well as individual amino acids and derivatives thereof, and mixtures of different amino acids. The molar weight of protein hydrolysates usable according to the invention is between 75 (the molar weight of glycine) and 200,000; the molar weight is preferably 75 to 50,000 daltons, and very particularly preferably 75 to 20,000 daltons.

The agent of the invention can include further at least one vitamin, provitamin, vitamin precursor, and/or a derivative thereof as a care substance. In this case, the vitamins, provitamins, and vitamin precursors of this kind that are usually assigned to the groups A, B, C, E, F, and H are preferred according to the invention.

Other care substances are panthenol, caffeine, nicotinamide, and sorbitol.

The agents of the invention, furthermore, can include at least one plant extract but also mono- or oligosaccharides and/or lipids as a care substance.

The composition of some particularly preferred cosmetic preparations of the invention can be obtained from the following tables (the data are given in percentages by weight, based on the total weight of the cosmetic agent, unless otherwise stated). The statements made previously regarding cosmetic preparations a) of the invention apply mutatis mutandis with respect to other preferred embodiments of these particularly preferred compositions.

| | Formula 1 | Formula 2 | Formula 3 | Formula 4 | Formula 5 |
|---|---|---|---|---|---|
| Ethanol a1) | 20 to 95 | 30 to 90 | 40 to 80 | 73 | 33 |
| Starch compound a2) | 5 to 80 | 10 to 70 | 20 to 60 | 26 | 66 |
| Optional additives | To 100 | To 100 | To 100 | To 100 | To 100 |
| | Formula 6 | Formula 7 | Formula 8 | Formula 9 | Formula 10 |
| Ethanol a1) | 20 to 95 | 30 to 90 | 40 to 80 | 73 | 33 |
| Oryza Sativa (Rice) | 5 to 80 | 10 to 70 | 20 to 60 | 26 | 66 |
| Starch | | | | | |
| Optional additives | To 100 | To 100 | To 100 | To 100 | To 100 |
| | Formula 11 | Formula 12 | Formula 13 | Formula 14 | Formula 15 |
| Ethanol a1) | 20 to 94 | 30 to 89 | 40 to 79 | 73 | 33 |
| Starch compound a2) | 5 to 79 | 10 to 69 | 20 to 59 | 26 | 66 |
| Cationic surfactant a3) | 0.01 to 4.0 | 0.05 to 2.0 | 0.1 to 1.0 | — | 0.03 |
| Optional additives | To 100 | To 100 | To 100 | To 100 | To 100 |
| | Formula 16 | Formula 17 | Formula 18 | Formula 19 | Formula 20 |
| Ethanol a1) | 20 to 94 | 30 to 89 | 40 to 79 | 73 | 33 |
| Oryza Sativa (Rice) | 5 to 79 | 10 to 69 | 20 to 59 | 26 | 66 |
| Starch | | | | | |
| Cationic surfactant a3) | 0.01 to 4.0 | 0.05 to 2.0 | 0.1 to 1.0 | — | 0.03 |
| Optional additives | To 100 | To 100 | To 100 | To 100 | To 100 |
| | Formula 21 | Formula 22 | Formula 23 | Formula 24 | Formula 25 |
| Ethanol a1) | 20 to 94 | 30 to 89 | 40 to 79 | 73 | 33 |
| Starch compound a2) | 5 to 79 | 10 to 69 | 20 to 59 | 26 | 66 |
| (C₁₂ to C₁₈) | 0.01 to 4.0 | 0.05 to 2.0 | 0.1 to 1.0 | — | 0.03 |
| alkyltrimethyl- | | | | | |
| ammonium salts | | | | | |
| Optional additives | To 100 | To 100 | To 100 | To 100 | To 100 |
| | Formula 26 | Formula 27 | Formula 28 | Formula 29 | Formula 30 |
| Ethanol a1) | 20 to 94 | 30 to 89 | 40 to 79 | 73 | 33 |
| Oryza Sativa (Rice) | 5 to 79 | 10 to 69 | 20 to 59 | 26 | 66 |
| Starch | | | | | |
| (C₁₂ to C₁₈) | 0.01 to 4.0 | 0.05 to 2.0 | 0.1 to 1.0 | — | 0.03 |
| alkyltrimethyl- | | | | | |
| ammonium salts | | | | | |
| Optional additives | To 100 | To 100 | To 100 | To 100 | To 100 |
| | Formula 31 | Formula 32 | Formula 33 | Formula 34 | Formula 35 |
| Ethanol a1) | 20 to 94 | 30 to 89 | 40 to 79 | 73 | 33 |
| Starch compound a2) | 5 to 79 | 10 to 69 | 20 to 59 | 26 | 66 |
| Cetyltrimethylammonium | 0.01 to 4.0 | 0.05 to 2.0 | 0.1 to 1.0 | — | 0.03 |
| salts | | | | | |
| Optional additives | To 100 | To 100 | To 100 | To 100 | To 100 |
| | Formula 36 | Formula 37 | Formula 38 | Formula 39 | Formula 40 |
| Ethanol a1) | 20 to 94 | 30 to 89 | 40 to 79 | 73 | 33 |
| Oryza Sativa (Rice) | 5 to 79 | 10 to 69 | 20 to 59 | 26 | 66 |
| Starch | | | | | |
| Cetyltrimethylammonium | 0.01 to 4.0 | 0.05 to 2.0 | 0.1 to 1.0 | — | 0.03 |
| salts | | | | | |
| Optional additives | To 100 | To 100 | To 100 | To 100 | To 100 |
| | Formula 41 | Formula 42 | Formula 43 | Formula 44 | Formula 45 |
| Ethanol a1) | 20 to 94 | 30 to 89 | 40 to 79 | 73 | 33 |
| Starch compound a2) | 5 to 79 | 10 to 69 | 20 to 59 | 26 | 66 |
| Hydrophobized metal | 0.01 to 3.0 | 0.05 to 2.5 | 0.1 to 2.0 | 1.4 | — |
| oxide powder a4) | | | | | |
| Optional additives | To 100 | To 100 | To 100 | To 100 | To 100 |
| | Formula 46 | Formula 47 | Formula 48 | Formula 49 | Formula 50 |
| Ethanol a1) | 20 to 94 | 30 to 89 | 40 to 79 | 73 | 33 |
| Oryza Sativa (Rice) | 5 to 79 | 10 to 69 | 20 to 59 | 26 | 66 |
| Starch | | | | | |
| Hydrophobized metal | 0.01 to 3.0 | 0.05 to 2.5 | 0.1 to 2.0 | 1.4 | — |
| oxide powder a4) | | | | | |
| Optional additives | To 100 | To 100 | To 100 | To 100 | To 100 |
| | Formula 51 | Formula 52 | Formula 53 | Formula 54 | Formula 55 |
| Ethanol a1) | 20 to 94 | 30 to 89 | 40 to 79 | 73 | 33 |
| Starch compound a2) | 5 to 79 | 10 to 69 | 20 to 59 | 26 | 66 |
| Silanized pyrogenic | 0.01 to 3.0 | 0.05 to 2.5 | 0.1 to 2.0 | 1.4 | — |
| silicon dioxide a4) | | | | | |
| Optional additives | To 100 | To 100 | To 100 | To 100 | To 100 |
| | Formula 56 | Formula 57 | Formula 58 | Formula 59 | Formula 60 |
| Ethanol a1) | 20 to 94 | 30 to 89 | 40 to 79 | 73 | 33 |
| Oryza Sativa (Rice) | 5 to 79 | 10 to 69 | 20 to 59 | 26 | 66 |
| Starch | | | | | |
| Silanized pyrogenic | 0.01 to 3.0 | 0.05 to 2.5 | 0.1 to 2.0 | 1.4 | — |
| silicon dioxide a4) | | | | | |
| Optional additives | To 100 | To 100 | To 100 | To 100 | To 100 |
| | Formula 61 | Formula 62 | Formula 63 | Formula 64 | Formula 65 |
| Ethanol a1) | 20 to 94 | 30 to 89 | 40 to 79 | 73 | 33 |
| Starch compound a2) | 5 to 79 | 10 to 69 | 20 to 59 | 26 | 66 |
| Silica Dimethyl | 0.01 to 3.0 | 0.05 to 2.5 | 0.1 to 2.0 | 1.4 | — |
| Silylate | | | | | |
| Optional additives | To 100 | To 100 | To 100 | To 100 | To 100 |
| | Formula 66 | Formula 67 | Formula 68 | Formula 69 | Formula 70 |
| Ethanol a1) | 20 to 94 | 30 to 89 | 40 to 79 | 73 | 33 |
| Oryza Sativa (Rice) | 5 to 79 | 10 to 69 | 20 to 59 | 26 | 66 |
| Starch | | | | | |
| Silica Dimethyl | 0.01 to 3.0 | 0.05 to 2.5 | 0.1 to 2.0 | 1.4 | — |
| Silylate | | | | | |
| Optional additives | To 100 | To 100 | To 100 | To 100 | To 100 |
| | Formula 71 | Formula 72 | Formula 73 | Formula 74 | Formula 75 |
| Ethanol a1) | 20 to 94 | 30 to 89 | 40 to 79 | 73 | 33 |
| Starch compound a2) | 5 to 79 | 10 to 69 | 20 to 59 | 26 | 66 |
| Cationic surfactant a3) | 0.01 to 4.0 | 0.05 to 2.0 | 0.1 to 1.0 | 0.02 | 0.05 |
| Silanized pyrogenic | 0.01 to 3.0 | 0.05 to 2.5 | 0.1 to 2.0 | 1.4 | 0.7 |
| silicon dioxide a4) | | | | | |
| Optional additives | To 100 | To 100 | To 100 | To 100 | To 100 |
| | Formula 77 | Formula 77 | Formula 78 | Formula 79 | Formula 80 |
| Ethanol a1) | 20 to 94 | 30 to 89 | 40 to 79 | 72 | 33 |
| Oryza Sativa (Rice) | 5 to 79 | 10 to 69 | 20 to 59 | 25 | 66 |
| Starch | | | | | |
| Cationic surfactant a3) | 0.01 to 4.0 | 0.05 to 2.0 | 0.1 to 1.0 | 0.02 | 0.05 |
| Hydrophobized metal | 0.01 to 3.0 | 0.05 to 2.5 | 0.1 to 2.0 | 1.4 | 0.7 |
| oxide powder a4) | | | | | |
| Optional additives | To 100 | To 100 | To 100 | To 100 | To 100 |
| | Formula 81 | Formula 82 | Formula 83 | Formula 84 | Formula 85 |
| Ethanol a1) | 20 to 94 | 30 to 89 | 40 to 79 | 73 | 33 |
| Starch compound a2) | 5 to 79 | 10 to 69 | 20 to 59 | 26 | 66 |
| (C₁₂ to C₁₈) | 0.01 to 4.0 | 0.05 to 2.0 | 0.1 to 1.0 | 0.02 | 0.05 |
| alkyltrimethyl- | | | | | |
| ammonium salts | | | | | |
| Silanized pyrogenic | 0.01 to 3.0 | 0.05 to 2.5 | 0.1 to 2.0 | 1.4 | 0.7 |
| silicon dioxide a4) | | | | | |
| Optional additives | To 100 | To 100 | To 100 | To 100 | To 100 |
| | Formula 86 | Formula 87 | Formula 88 | Formula 89 | Formula 90 |
| Ethanol a1) | 20 to 94 | 30 to 89 | 40 to 79 | 73 | 33 |
| Oryza Sativa (Rice) | 5 to 79 | 10 to 69 | 20 to 59 | 26 | 66 |
| Starch | | | | | |
| (C₁₂ to C₁₈) | 0.01 to 4.0 | 0.05 to 2.0 | 0.1 to 1.0 | 0.02 | 0.05 |
| alkyltrimethyl- | | | | | |
| ammonium salts | | | | | |
| Silanized pyrogenic | 0.01 to 3.0 | 0.05 to 2.5 | 0.1 to 2.0 | 1.4 | 0.7 |
| silicon dioxide a4) | | | | | |
| Optional additives | To 100 | To 100 | To 100 | To 100 | To 100 |
| | Formula 91 | Formula 92 | Formula 93 | Formula 94 | Formula 95 |
| Ethanol a1) | 20 to 94 | 30 to 89 | 40 to 79 | 73 | 33 |
| Starch compound a2) | 5 to 79 | 10 to 69 | 20 to 59 | 26 | 66 |
| Cetyltrimethylammonium | 0.01 to 4.0 | 0.05 to 2.0 | 0.1 to 1.0 | 0.02 | 0.05 |
| salts | | | | | |
| Silica Dimethyl Silylate | 0.01 to 3.0 | 0.05 to 2.5 | 0.1 to 2.0 | 1.4 | 0.7 |
| Optional additives | To 100 | To 100 | To 100 | To 100 | To 100 |
| | Formula 96 | Formula 97 | Formula 98 | Formula 99 | Formula 100 |
| Ethanol a1) | 20 to 94 | 30 to 89 | 40 to 79 | 73 | 33 |
| Oryza Sativa (Rice) | 5 to 79 | 10 to 69 | 20 to 59 | 26 | 66 |
| Starch | | | | | |
| Cetyltrimethylammonium | 0.01 to 4.0 | 0.05 to 2.0 | 0.1 to 1.0 | 0.02 | 0.05 |
| salts | | | | | |
| Silica Dimethyl Silylate | 0.01 to 3.0 | 0.05 to 2.5 | 0.1 to 2.0 | 1.4 | 0.7 |
| Optional additives | To 100 | To 100 | To 100 | To 100 | To 100 |

Preferred cosmetic preparations a), based on their total weight, consist of at least 80% by weight, preferably at least 90% by weight, and in particular at least 97% by weight of components a1) and a2). If active and auxiliary substances from the group of cationic surfactants a3) and/or the hydrophobically modified metal oxide powder a4) are used in cosmetic preparations a) of the invention, thus it is preferable that cosmetic preparation a), based on its total weight, consists of at least 80% by weight, preferably at least 90% by weight, and in particular at least 97% by weight of components a1), a2) and component a3) and/or a4).

As set forth above, cosmetic preparations a) of the invention are suitable in a particular way for application by means of a device for flash evaporation. A further subject of the present invention, therefore, is the use of a cosmetic preparation a) including, based on its total weight,
a1) 20 to 95% by weight of ethanol,
a2) 5 to 80% by weight of at least one starch compound,
   as a process material in a device for flash evaporation.

The subject of the present invention, moreover, is the use of a product of the invention for treating keratin-containing fibers, in particular human hair, with a cosmetic preparation a) or for cleaning keratin-containing fibers, in particular human hair.

A method for the temporary shaping of keratin-containing fibers, in particular human hair, in which the keratin-containing fibers are acted upon by a device for flash evaporation with a cosmetic preparation a) including, based on its total weight,
a1) 20 to 95% by weight of ethanol,
a2) 5 to 80% by weight of at least one starch compound,
   is a further subject of the present invention. Cosmetic preparation a) is converted by the device for flash evaporation preferably into a spray mist, which subsequently acts upon the keratin-containing fibers.

So as to achieve a sufficient spray effect, cosmetic preparation a) is preferably heated to temperatures above the boiling point of the polar solvent or solvent mixture included in cosmetic preparation a).

If the polar solvent is water or solvent mixtures with a proportion of water above 50% by weight (based on the total weight of the solvent mixture), the cosmetic preparation is preferably heated to temperatures above 100° C., preferably to temperatures of 100° C. and 240° C., particularly preferably to temperatures of 140° C. to 160° C.

The excess pressure, achieved by heating cosmetic preparation a), in the cases in which the polar solvent is water or solvent mixtures with a proportion of water above 50% by weight (based on the total weight of the solvent mixture), is preferably between 1.1 and 8 bar, preferably between 1.2 and 4 bar.

A preferred subject of the invention is a method for changing the color of keratin-containing fibers, in particular human hair, in which the keratin-containing fibers are acted upon by a device for flash evaporation with a cosmetic preparation a) including, based on its total weight,
a1) 20 to 95% by weight of ethanol,
a2) 5 to 80% by weight of at least one starch compound,
wherein
   a partial amount of cosmetic preparation a) located in a reservoir is transferred from this reservoir, in whose interior a pressure corresponding to the ambient pressure predominates, into a container b1);
   thereafter the access between the reservoir and container b1) is interrupted by a structural element for flow regulation by which the flow of cosmetic preparation a) from the reservoir into container b1) can be interrupted;
   thereafter cosmetic preparation a) located in container b1), closed off from the environment, is heated by a heating device, so that the pressure in the interior of container b1) increases to values above the ambient pressure, preferably to values between 1.1 and 8 bar, in particular to values between 1.2 and 4 bar;
   thereafter container b1), having a pressure above the ambient pressure, is opened in a way that releases the exit of at least one partial amount, preferably at least 50% by weight, preferably at least 80% by weight, and in particular at least 90% by weight of the cosmetic preparation, located in container b1), from container b1) into the environment with a reduction of the pressure predominating in container b1) at the time the container is opened.

The release of cosmetic preparation a) into the environment occurs preferably with the formation of a spray mist of cosmetic preparation a).

Cosmetic preparation a) released from container b1) is preferably applied to keratinic fibers, in particular human hair.

Methods during which the cosmetic preparation released from container b1) is passed through a nozzle before action upon the keratinic fibers are particularly preferred.

The statements made in regard to cosmetic preparations a) of the invention and the device for flash evaporation b) apply mutatis mutandis with respect to further preferred embodiments of the uses of the invention and the method of the invention.

The agents, uses, and methods of the invention and some of their preferred embodiments are characterized in summary by the following points:

A cosmetic product comprising
a) a cosmetic preparation including, based on its total weight,
   a1) 20 to 95% by weight of ethanol,
   a2) 5 to 80% by weight of at least one starch compound;
b) a device for the flash evaporation of cosmetic preparation a).

The cosmetic product according to point 1, characterized in that the device for flash evaporation comprises a container b1) and a heating device b2) and is designed such that
cosmetic preparation a) can be taken up in the interior space of container b1),
the interior space of container b1), said space filled at least partially with cosmetic preparation a), can be closed,
cosmetic preparation a) can be heated in the closed interior space of container b1) by means of heating device b2) with an increase in pressure,
the heated cosmetic preparation a) can be released from the interior space of container b1) into the environment with a reduction in pressure.

The cosmetic product according to one of the preceding points, characterized in that the weight proportion of ethanol a1) in terms of the total weight of cosmetic preparation a) is 30 to 90% by weight, preferably 40 to 80% by weight.

The cosmetic product according to one of the preceding points, characterized in that the weight proportion of the starch compound in terms of the total weight of cosmetic preparation a) is 10 to 70% by weight, preferably 20 to 60% by weight.

The cosmetic product according to one of the preceding points, characterized in that starch compound a2) is selected from the group of, optionally modified, polycondensation products of D-glucose obtained from the starch of potatoes, corn, rice, peas, acorns, chestnuts, barley, wheat, bananas, sago, millet, sorghum, oats, barley, rye, beans, sweet potato, arrowroot, or cassava.

The cosmetic product according to one of the preceding points, characterized in that cosmetic preparation a), based on its total weight, includes 0.01 to 4.0% by weight, preferably 0.05 to 2.0% by weight, and in particular 0.1 to 1.0% by weight of a cationic surfactant a3).

The cosmetic product according to point 5, characterized in that cationic surfactant a3) is selected from the groups of quaternary ammonium compounds, esterquats, and amidoamines, preferably from the group of quaternary ammonium compounds, in particular from the group of (C₁₂ to C₁₈) alkyltrimethylammonium salts.

The cosmetic product according to one of the preceding points, characterized in that cosmetic preparation a), based on its total weight, includes 0.01 to 3.0% by weight, preferably 0.05 to 2.5% by weight, and in particular 0.1 to 2.0% by weight of a hydrophobically modified metal oxide powder a4).

The cosmetic agent according to point 8, characterized in that the hydrophobically modified metal oxide powder a4) is selected from the group of hydrophobized silicates obtained by silanization of pyrogenic silicon dioxide.

The cosmetic product according to point 8, characterized in that the hydrophobically modified metal oxide powder a4) is selected from the compounds with the INCI names: Silica Dimethyl Silylate (e.g., Aerosil R792, Aerosil R794), Silica Dimethicone Silylate (e.g., Aerosil R202), and Silica Silylate (e.g., Aerosil R805, Aerosil R812, Aerosil R816), preferably from the compounds with the INCI name Silica Dimethyl Silylate.

The cosmetic product according to one of the preceding points, characterized in that cosmetic preparation a), based on its total weight, consists of at least 80% by weight, preferably at least 90% by weight, and in particular at least 97% by weight of components a1) and a2).

The cosmetic product according to one of the preceding points, characterized in that cosmetic preparation a), based on its total weight, consists of at least 80% by weight, preferably at least 90% by weight, and in particular at least 97% by weight of components a1), a2) and component a3) and/or a4).

Use of a cosmetic preparation a) including, based on its total weight,
a1) 20 to 98% by weight of ethanol,
a2) 5 to 80% by weight of at least one starch compound,
   as a process material in a device for flash evaporation.

The use of a product according to one of points 1 to 12 for treating keratin-containing fibers, in particular human hair, with a cosmetic preparation a).

The use of a product according to one of points 1 to 12 for cleaning keratin-containing fibers, in particular human hair.

A method for the treatment of keratin-containing fibers, in particular human hair, in which the keratin-containing fibers are acted upon by a device for flash evaporation with a cosmetic preparation a) including, based on its total weight,
a1) 20 to 98% by weight of ethanol,
a2) 5 to 80% by weight of at least one starch compound.

The method according to point 16, characterized in that
a partial amount of cosmetic preparation a) located in a reservoir is transferred from this reservoir, in whose interior a pressure corresponding to the ambient pressure predominates, into a container b1);
thereafter the access between the reservoir and container b1) is interrupted by a structural element for flow regulation by which the flow of cosmetic preparation a) from the reservoir into container b1) can be interrupted;
thereafter cosmetic preparation a) located in container b1), closed off from the environment, is heated by a heating device, so that the pressure in the interior of container b1) increases to values above the ambient pressure, preferably to values between 1.1 and 8 bar, in particular to values between 1.2 and 4 bar;
thereafter container b1), having a pressure above the ambient pressure, is opened in a way that releases the exit of at least one partial amount, preferably at least 50% by weight, preferably at least 80% by weight, and in particular at least 90% by weight of the cosmetic preparation, located in container b1), from container b1) into the environment with a reduction of the pressure predominating in container b1) at the time the container is opened.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic product, comprising:
a) a cosmetic preparation including, based on its total weight,
a1) 20 to 95% by weight of ethanol,
a2) 5 to 80% by weight of at least one starch compound;
b) a device for the flash evaporation of cosmetic preparation a).

2. The cosmetic product according to claim 1, wherein the device for flash evaporation comprises a container b1) and a heating device b2) and is configured such that
cosmetic preparation a) can be taken up in the interior space of container b1),
the interior space of container b1), said space being filled at least partially with cosmetic preparation a), can be closed,
cosmetic preparation a) can be heated in the closed interior space of container b1) by means of heating device b2) with an increase in pressure,
the heated cosmetic preparation a) can be released from the interior space of container b1) into the environment with a reduction in pressure.

3. The cosmetic product according claim 1, wherein the weight proportion of ethanol a1) in terms of the total weight of cosmetic preparation a) is 30 to 90% by weight.

4. The cosmetic product according to claim 1, wherein the weight proportion of ethanol a1) in terms of the total weight of cosmetic preparation a) is 40 to 80% by weight.

5. The cosmetic product according to claim 1, wherein the weight proportion of the starch compound in terms of the total weight of cosmetic preparation a) is 10 to 70% by weight.

6. The cosmetic product according to claim 1, wherein the weight proportion of the starch compound in terms of total weight of cosmetic preparation a) is 20 to 60% by weight.

7. The cosmetic product according to claim 1, wherein the starch compound a2) is an optionally modified polycondensation products of D-glucose obtained from the starch of potatoes, corn, rice, peas, acorns, chestnuts, barley, wheat, bananas, sago, millet, sorghum, oats, barley, rye, beans, sweet potato, arrowroot, or cassava.

8. The cosmetic product according to claim 1, wherein the cosmetic preparation a), based on its total weight, includes 0.01 to 4.0% by weight of a cationic surfactant a3).

9. The cosmetic product according to claim 1, wherein the cosmetic preparation a), based on its total weight, includes 0.05 to 2.0% by weight of a cationic surfactant a3).

10. The cosmetic product according to claim 1, wherein the cosmetic preparation a), based on its total weight, includes 0.01 to 1.0% by weight of a cationic surfactant a3).

11. The cosmetic product according to claim 1, wherein the cosmetic preparation a), based on its total weight, includes 0.01 to 3.0% by weight of a hydrophobically modified metal oxide powder a4).

12. The cosmetic product according to claim 1, wherein the cosmetic preparation a), based on its total weight, includes 0.05 to 2.5% by weight of a hydrophobically modified metal oxide powder a4).

13. The cosmetic product according to claim 1, wherein the cosmetic preparation a), based on its total weight, includes 0.1 to 2.0% by weight of a hydrophobically modified metal oxide powder a4).

14. A method for cleaning keratin-containing fibers, comprising:
using the cosmetic product of claim 1 to apply the cosmetic preparation a) to keratin-containing fibers.
